(19) United States
(12) Patent Application Publication
(10) Pub. No.: US 2023/0017926 A1
(43) Pub. Date: Jan. 19, 2023

(54) Colorimetric Based Gastric Tube Placement Verification System and Method

(71) Applicant: Charles Kim Gilmartin, San Anselmo, CA (US)

(72) Inventor: Charles Kim Gilmartin, San Anselmo, CA (US)

(21) Appl. No.: 17/943,371

(22) Filed: Sep. 13, 2022

Related U.S. Application Data
(63) Continuation of application No. PCT/US21/22438, filed on Mar. 15, 2021.
(60) Provisional application No. 62/988,925, filed on Mar. 13, 2020.

(51) Int. Cl. A61J 15/00

(52) U.S. Cl. CPC A61J 15/0084; A61J 2200/70

(57) ABSTRACT

A method of gastric tube placement verification comprising the steps of: Inserting a gastric tube within the patient; Providing a colorimetric based gastric tube placement verification system for a patient gastric tube including i) a housing configured to be coupled to the gastric tube whereby stomach content aspirate can flow through an internal passage of the housing; and ii) at least one colorimetric based sensor within the housing and configured to come into contact with the patient stomach content aspirate, the least one colorimetric based sensor configures to detect a first gastric acid; Coupling the housing of the colorimetric based gastric tube placement verification system to a proximal end of the gastric tube; Aspirating stomach content of the patient whereby stomach aspirate can flow through an internal passage of the housing; and Visually inspecting at least one colorimetric based sensor within the housing for verification of proper gastric tube placement.

### RELATED APPLICATIONS

This application is a continuation of International Patent Application Serial Number PCT/US2021/022438 filed Mar. 15, 2021 and published Sep. 16, 2021 as Publication Number WO 2021-184023 titled “Colorimetric Based Gastric Tube Placement Verification System and Method” which application and publication are incorporated herein in their entireties.

International Patent Application Serial Number PCT/US2021/022438 claims priority to U.S. Patent Application Ser. No. 62/988,925 filed Mar. 13, 2020 titled “COLORIMETRIC PAPER BASED CONFIRMATION OF PROPER PLACEMENT OF OROGASTRIC/NASOGASTRIC AND FEEDING TUBES” which application is incorporated herein in its entirety.

### BACKGROUND INFORMATION

### 1. Field of the Invention

The present invention relates to a colorimetric based gastric tube placement verification system improving confirmation rates for the proper placement of gastric tubes, such as oro-gastric, nasogastric and feeding tubes, and an associated method of use.

### 2. Background Information

A conventional gastric tube 100 in a patient 10 is shown in FIG. 1 for reference. Gastric tubes 100 as a class typically include oro-gastric, nasogastric and feeding tubes. Gastric tubes 100 are ubiquitous and are used routinely in hospitals and outpatient surgery centers and other various locations. Gastric tubes 100 are small tubes placed either through the nose or mouth through the esophagus 14 and often end with the tip in the stomach 14 (although certain applications can extend further). Gastric tubes 100 are typically used for feeding, medication administration, or removal of contents from the stomach via aspiration, suction, or gravity drainage.

Currently, placement of gastric tubes 100 is accomplished, for a nasogastric tube 100 implementation, by using the nostril with the largest opening to insert the nasogastric tube 100 down the back of the nostril to the nasopharynx. The medical professional will generally ask the patient 10 to swallow once the tube 100 enters the nasopharynx. If the patient 10 is not able to mimic the swallowing action, the caregiver will often ask the patient 10 to sip water. For oro-gastric tubes 100 the technique is similar although placement starts in the oral cavity.

Complications that result from improper OG/NG tube 100 placement including pneumothorax, pulmonary hemorrhage, pleural effusion, empyema, trauma injuries, abscess formation, nose bleeds, asphyxia, secondary infections, pneumonitis, and development of tracheal-esophageal fistula. Other complications that can occur from improper tube 100 placement include tube migration, perforation of the tube 100, and the tube obstruction.

The ability to safely assess gastric tube 100 placement is a key skill that most medical professionals are required to learn. It is essential that the medical professional apply a systematic approach to such assessments as incorrect gastric tube 100 placement can result in life threatening complications. Historically, nurses or other qualified healthcare professionals can verify the placement of the OG/NG tube 100 by performing two of the following methods: asking the patient to hum or talk (coughing or choking means the tube 100 is properly placed); use an irrigation syringe to aspire gastric contents; chest xray, lower the open end or proximal of the OG/NG tube 100 into a cup of water (bubbles indicate that the tube 100 is in place); or place a stethoscope over the patient's epigastrium while a 30 cclml bolus using an irrigation syringe (the air enters the stomach 14 when a whooshing sound is heard).

Methods that are most commonly used for confirming or verifying gastric tube 100 placement include: measuring the pH of aspirate using pH indicator strips or radiography (e.g. chest x-ray). Although other methods are discussed, currently these are the two modalities that are the most readily accepted. In the PH testing verification methodology, after a gastric tube 100 has been inserted, it is common practice to attempt to obtain a aspirate which then can have its pH checked. The idea is that gastric contents normally have a low pH (1.5-3.5) and therefore any aspirate that has a pH this low is likely to be located in the stomach 14 and unlikely to be located elsewhere (e.g. the respiratory tract). Aspiration and PH checking can therefore potentially be used as a method of confirming safe nasogastric placement without the additional need of chest X Ray if the pH is within a safe range (0-5.5). Local guidelines, however, can differ in terms of the acceptable pH range for confirming gastric tube 100 placement and some hospitals may require chest X-Rays for all patients 10, regardless of pH aspirate. Further hindering the PH testing method is that stomach pH can be altered by medications (e.g. proton pump inhibitors) and by the frequency of feeds.

Publication Number WO 2021-184023 identified U.S. Pat. No. 8,361,041 from the University of Utah Research Foundation; Korean Patent Publication 2016-0094397 and the article Susan Roberts, et al Devices and Techniques for Bedside Enteral Feeding Tube Placement. Nutrition in Clinical Practice 22:412-, August 2007 as being of relevant to the general state of the art to the present invention.

There remains a need for an accurate, efficient and cost-effective method for proper placement confirmation or verification of gastric tubes 100.

### SUMMARY OF INVENTION

The colorimetric based gastric tube placement verification system and associated method of the present invention gives a safe and cost-effective method for proper tube placement while reducing morbidity and mortality of patients.

One aspect of the invention provides a method of gastric tube placement verification comprising the steps of: Inserting a gastric tube within the patient; Providing a colorimetric based gastric tube placement verification system for a patient gastric tube including i) a housing configured to be coupled to the gastric tube whereby stomach content aspirate can flow through an internal passage of the housing; and ii) at least one colorimetric based sensor within the housing and configured to come into contact with the patient stomach content aspirate, the least one colorimetric based sensor configures to detect a first gastric acid; Coupling the housing of the colorimetric based gastric tube placement verification system to a proximal end of the gastric tube; Aspirating stomach content of the patient whereby stomach aspirate can flow through an internal passage of the housing; and Visually inspecting at least one colorimetric based sensor within the housing for verification of proper gastric tube placement.

One aspect of the invention provides a colorimetric based gastric tube placement verification system for a patient gastric tube comprising: a housing configured to be coupled to the gastric whereby stomach content aspirate can flow through an internal passage of the housing; and at least one colorimetric based sensor within the housing and configured to come into contact with the patient stomach content aspirate, the least one colorimetric based sensor configures to detect a first gastric acid.

One aspect of the invention provides a colorimetric based gastric tube placement verification system for a patient gastric tube comprising: a housing configured to be coupled to the gastric whereby stomach content aspirate can flow through an internal passage of the housing; and a colorimetric sensor array comprising at least two colorimetric based sensors within the housing and configured to come into contact with the patient stomach content aspirate, including i) a sensor for a first gastric acid, and ii) a sensor for a second gastric acid different from the first gastric acid.

These and other advantages of the present invention are described in connection with the following figures in which like elements use like reference numerals throughout.

### BRIEF DESCRIPTION OF THE DRAWINGS

FIG. 1 is a schematic view of a conventional gastric tube, namely a nasogastric tube, as known in the art;

FIG. 2A is a perspective view of a colorimetric based gastric tube placement verification system according to one embodiment of the present invention;

FIG. 2B is an exploded perspective view of the colorimetric based gastric tube placement verification system of FIG. 2A;

FIG. 2C is a perspective view of the colorimetric based gastric tube placement verification system of FIG. 2A with a lower housing shown in phantom for clarity;

FIG. 2D is an elevation section view of the lower housing of the colorimetric based gastric tube placement verification system of FIG. 2A;

FIG. 2E is a perspective view of an upper housing of the colorimetric based gastric tube placement verification system of FIG. 2A;

FIG. 3A is a perspective view of a colorimetric based colorimetric based gastric tube placement verification system according to another embodiment of the present invention;

FIG. 3B is an exploded perspective view of the colorimetric based gastric tube placement verification system of FIG. 3A;

FIG. 3C is a perspective section view of the colorimetric based gastric tube placement verification system of FIG. 3A;

FIG. 3D is a perspective view of an upper housing of the colorimetric based gastric tube placement verification system of FIG. 3A;

FIG. 3E is a perspective view of a lower housing of the colorimetric based gastric tube placement verification system of FIG. 3A;

FIG. 4A is a perspective view of a colorimetric based gastric tube placement verification system according to another embodiment of the present invention;

FIG. 4B is an exploded perspective view of the colorimetric based gastric tube placement verification system of FIG. 4A;

FIG. 5A is a perspective view of a colorimetric based gastric tube placement verification system according to another embodiment of the present invention; and

FIG. 5B is an exploded perspective view of the colorimetric based gastric tube placement verification system of FIG. 5A.

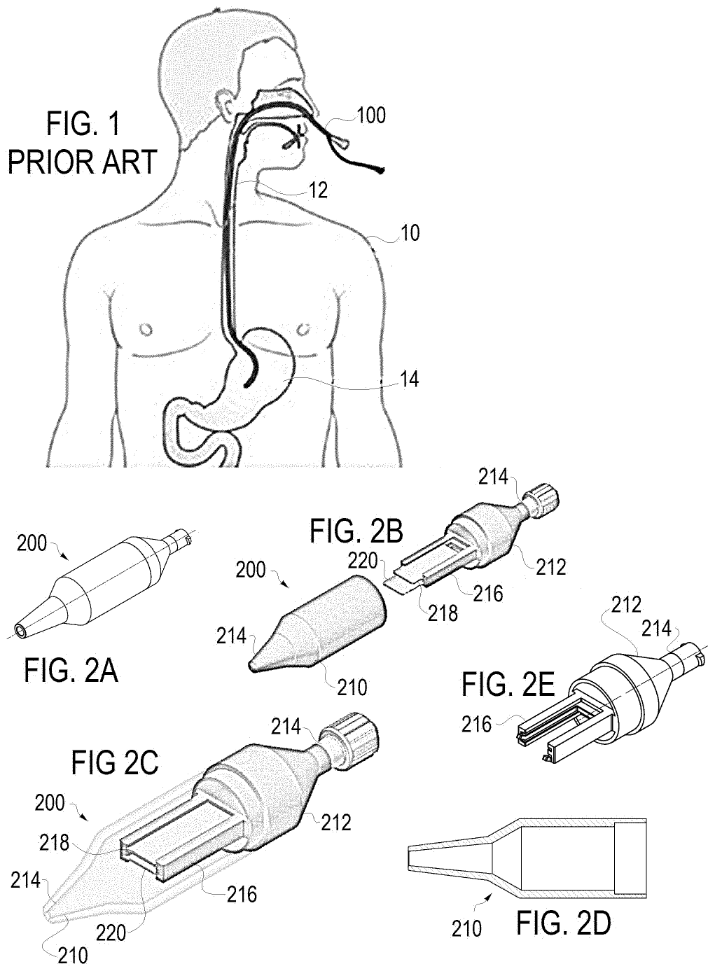

### DESCRIPTION OF THE PREFERRED EMBODIMENTS

One aspect of this invention is directed to an integrated HCl and butyric acid colorimetric based detection system 200, 300, which would allow for a quick, effective and safe placement verification system for orogastric, nasogastric and feeding tubes 100.

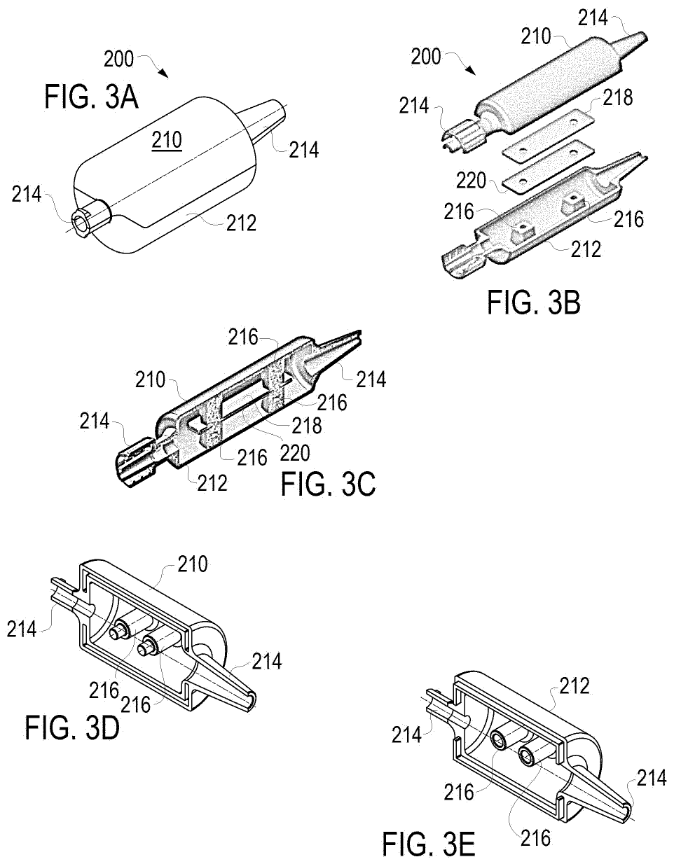

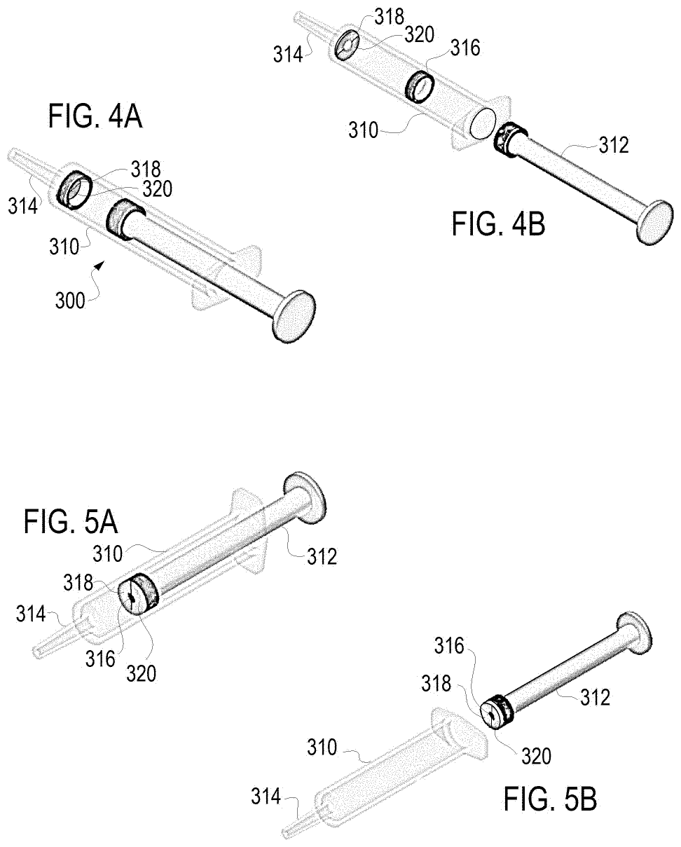

FIGS. 2A-E illustrate a colorimetric based gastric tube placement verification system 200 according to one embodiment of the present invention, FIGS. 3A-E illustrate a colorimetric based gastric tube placement verification system 200 according to another embodiment of the present invention, FIGS. 4A-B illustrate a syringe type colorimetric based gastric tube placement verification system 300 according to another embodiment of the present invention, and FIGS. 5A-B illustrate a syringe type colorimetric based gastric tube placement verification system 300 according to another embodiment of the present invention.

Each system 200 and 300 is used in a method of gastric tube 100 placement verification comprising the steps of: Inserting a gastric tube 100 within the patient 10 in a conventional fashion; Providing a colorimetric based gastric tube placement verification system 200, 300 for a patient gastric tube 100 including i) a housing 210, 212, 310, 312 configured to be coupled to the gastric tube 100 whereby stomach 16 content aspirate can flow through an internal passage of the housing 210, 212, 310, 312; and ii) at least one colorimetric based sensor 218, 220, 318, 320 within the housing 210, 212, 310, 312 and configured to come into contact with the patient stomach content aspirate, the least one colorimetric based sensor 218, 220, 318, 320 configures to detect a first gastric acid, such as HCL or Butyric Acid; Coupling the housing of the colorimetric based gastric tube placement verification system 200, 300 to a proximal end of the gastric tube 100; Aspirating stomach 14 content of the patient 10 whereby stomach aspirate can flow through an internal passage of the housing 210, 212, 310, 312; and Visually inspecting at least one colorimetric based sensor 218, 220, 318, 320 within the housing 210, 212, 310, 312 for verification of proper gastric tube 100 placement.

HCl, Hydrochloric acid or muriatic acid, is a primary acid found in the stomach and in stomach aspirate. Hydrochloric acid or muriatic acid is a colorless inorganic chemical system with the formula HCl. Hydrochloric acid has a distinctive pungent smell. It is classified as a strongly acidic acid and can attack the skin over a wide composition range, since hydrogen chloride completely disassociates in aqueous solution. Hydrochloric acid is the simplest chlorine-based acid system. It is the solution of hydrogen chloride and water in a variety of other chemical species including hydronium and chloride ions. It is a naturally occurring component of the gastric acid produced in the digestive system of most animal species, including humans.

Butyric acid, also known under the systematic name butanoic acid is a carboxylic acid with the structural formula CH3CH2CH2C02H. Classified as a carboxylic acid, it is oily, colorless liquid that is soluble in water, ethanol, and ether. Isobutyric acid is an isomer. Butyric acid is a carboxylic acid found in rancid butter, parmesan cheese, and vomit, and has an unpleasant odor and acrid taste, with a sweetish aftertaste (similar to ether). Butyric acid is a fatty acid occurring in the form of esters in animal fats and plant oils.

As noted above, FIGS. 2A-E illustrate a colorimetric based gastric tube placement verification system 200 according to one embodiment of the present invention. The housing 210 and 212 is formed of transparent plastic and is formed of an upper housing 210 and a lower housing 212 that can be glued together. The housing defines an internal passage there through with couplings 214 on the housing 210 and 212 to couple to the proximal end of the tube 100 and other components upstream thereof, like a suction pump. The connections or coupling 214 may be a luer lock attachment or other conventional attachment components common in the medical fields.

The upper housing includes sensor supports 216 in the form or slots receiving two colorimetric sensors 218 and 220 therein detecting HCL and Butyric Acid, respectively. The colorimetric sensors 218 and 220 form an optical chemical sensor array and each are formed as a substrate, generally filter paper, impregnated with an indicator that visibly changes color via a chemical reaction in the presence of a present amount of the sensed target substrate. See for example Johnson Test Paper, CBRNE Tech Index (http://www.cbrnetechindex.com/Chemical-Detection/Technology-CD/Colorimetric-CD-T), and Millipore Sigma. For the purpose of the present invention the colorimetric sensors 218 and 220 will exhibit a color change generally in less than 2 seconds when exposed to the parameter of interest. For example, the colorimetric paper from Johnson Test paper forming the HCL sensor 218 changes color from blue to pink in the presence of HCl, with the sensitivity of the paper specified to be 0.5 ppm. The test or filter paper forming the Butyric Acid sensor 220 changes color in the presence of Buteric Acid, with the sensitivity of the paper specified to be 0.5 ppm. The sensor 220 should has a base color and a triggered color different from the sensor 218.

As noted above, FIGS. 3A-E illustrate a colorimetric based gastric tube placement verification system 200 according to another embodiment of the present invention. Analogous to the embodiment of FIGS. 2A-E, the housing 210 and 212 of the embodiment of FIGS. 3A-E is formed of transparent plastic and is formed of an upper housing 210 and a lower housing 212 that can be glued together. The housing defines an internal passage there through, with couplings 214 on the housing 210 and 212 to couple to the proximal end of the tube 100 and other components upstream thereof, like a suction pump. The connections or coupling 214 may be a luer lock attachment or other conventional attachment components common in the medical fields.

The upper housing includes sensor supports 216 which here are in the form of posts, again, receiving two colorimetric sensors 218 and 220 therein detecting HCL and Butyric Acid, respectively. The sensors 218 and 220 in FIGS. 3A-E are the same as used in the embodiment of FIGS. 2A-E except they are sized to be received on the posts 26 with matching holes.

In summary, the system 200 of FIGS. 2A-E uses a clear housing 210, 212 with the colorimetric paper of sensors 218 and 220 attached onto/into two slots formed by sensor holder 216 that will allow for aspirate to pass over the colorimetric paper sensors 218 and 220 when suction is applied via syringe or mechanical suction. The clear housing 210 and 212 allows for quick visual confirmation of any color changes in the colorimetric paper of sensors 218 and 220 to verify proper placement of the gastric tube 100. Similarly, the system 200 of FIGS. 3A-E uses a clear housing 210, 212 for easy visual detection/confirmation of color change of either or both of the included colorimetric paper sensors 220 and 218 (i.e. butyric acid and HCl).

The HCL sensor 218 and the butyric acid sensor 220 in the embodiments of FIGS. 2A-E and 3A-E above operate on different parameters to achieve the same purpose. In practice it is expected that there will be some situations in which the HCL sensor 218 operates faster at detecting stomach acids than the butyric acid sensor 220, and vice versa. The faster detection of one gastric acid over the other may have population dependent parameters, however including both within the system 220 improves response times as well as system efficacy.

It is possible to add a third colorimetric sensor in the form of a PH colorimetric sensor which will effectively respond to the low PH of gastric acids. Adding a third sensor requires placement in a location that is visible through the housing 210 and 212. The normal pH range for stomach acid is between 1.5 and 3.5. The trigger point of the PH sensor may be selected within a range of intra-gastric PH ranges for humans. See pH dependence of acid secretion and gastrin release in normal and ulcer subjects. Walsh J H, Richardson C T, Fordtran J S J Clin Invest. 1975 March; 55(3):462-8. One class of PH colorimetric sensor 20 is a graphene oxide based sensor that exhibits distinctive color response. See Efficient Colorimetric pH Sensor Based on Responsive Polymer—Quantum Dot Integrated Graphene Oxide, Kwanyeol Paek, Hyunseung Yang, Junhyuk Lee, Junwoo Park, and Bumjoon J. Kim ACS Nano 2014 8 (3), 2848-2856. As noted above the PH of the stomach 14 of the patient 10 can change such that the PH sensor should only supplement the remaining colorimetric sensor. Further the color of the PH sensor, if present, should differ from that of the sensors 218 and 220.

FIGS. 4A-B illustrate a syringe type colorimetric based gastric tube placement verification system 300 according to another embodiment of the present invention. The housing 310 is formed of transparent plastic and is formed as a syringe barrel of an aspirational syringe. The housing can be considered to include the plunger 312 of the syringe housing 310. The syringe barrel housing 310 includes a coupling 314 to couple to the proximal end of the tube 100 for aspiration by withdrawal of the plunger 312 in conventional fashion. The housing 310 receives two colorimetric sensors 318 and 320 therein detecting HCL and Butyric Acid, respectively. The colorimetric sensors 318 and 320 form an optical chemical sensor array combined in the form of an annular disc. Each sensor 318 and 320 are each formed analogous to sensors 218 and 220, namely as a substrate, generally filter paper, impregnated with an indicator that visibly changes color via a chemical reaction in the presence of a present amount of the sensed target substrate. The optical chemical sensor array can be held in place with a friction fit rubber ring 316, which can avoid the use of glues that would hinder the operation of the sensors 318 and 320.

FIGS. 5A-B illustrate a syringe type colorimetric based gastric tube placement verification system 300 according to another embodiment of the present invention which is analogous to the system 300 of FIGS. 4A-B. The housing 310 is formed of transparent plastic and is formed as a syringe barrel of an aspirational syringe. The housing can be considered to include the plunger 312 of the syringe housing 310. The syringe barrel housing 310 includes a coupling 314 to couple to the proximal end of the tube 100 for aspiration by withdrawal of the plunger 312 in conventional fashion. The housing 310 receives two colorimetric sensors 318 and 320 on the end of plunger 312 therein detecting HCL and Butyric Acid, respectively. The colorimetric sensors 318 and 320 form an optical chemical sensor array combined in the form of an annular disc. In this embodiment the sensors 318 and 320 may be secured onto the plunger 312 with a mechanical fastener 316 (e.g. screw or bolt) to minimize the use of glue.

The system 300 of FIGS. 4A-B and 5A-B utilize the aspiration syringe of housing 310 and plunger 312 (or upper and lower housing 310 and 312) to draw aspirant through the gastric tube 100, allowing for contact of the fluid aspirant with the colorimetric paper sensors 318 and 320 in the syringe or housing 310.

As noted above Butyric acid/HCl detection in system 200 or 300 can be achieved through direct contact of sensors 218, 220, 318, 320 with aspirate after connection to the proximal end of the OG/NG gastric tube 100. Attaching the detection device or system 200 or 300 to the proximal end of the OG/NG gastric tube 100 and then either attaching the opposite end to a suction device and/or a aspirating syringe, the aspirate will come in contact with the colorimetric paper sensor 218, 220, 318, 320 giving a positive color reaction when in the presence of butyric acid and or HCl.

The system 200 and 300 show several attachment sites for the colorimetric paper sensors 218, 220, 318, 320. The colorimetric paper sensors 218, 220, 318, 320 could be attached to the sides of the housing 210, 310 that will be exposed to the stomach aspirate as it is suctioned or drawn through the system 200 or 300. These housing could have colorimetric paper sensor receiving slots within the housing with perforations or small holes which allows for liquid contact with the colorimetric paper sensors. These access sites could have small access holes or vertical openings within the internal casing. Using the butyric acid/HCl colorimetric based detection system 200 or 300 for the confirmation of proper placement of gastric tubes 100, it allows for a far greater level of certainty rather than the current methods for detection of proper placement. The apparatus 200 or 300 can detect butyric acid and HCL in quantities of parts per millions with high specificity and selectivity. The apparatus 200 or 300 is configured to adapt to current orogastric/nasogastric and feeding tubes 100.

Presented herein are a few versions of butyric acid, HCl (and or pH) colorimetric sensor 218, 220, 318 and 320 designs and described operations. Note that the above descriptions are not exhaustive, and do not restrict the applicability of the approach presented here and are meant to serve as illustrations. Further embodiments of the apparatuses 200, 300 will become obvious after study of the apparatuses 200 and 300 presented herein by persons with experience in the art or area

While the invention has been shown in several particular embodiments it should be clear that various modifications may be made to the present invention without departing from the spirit and scope thereof. The scope of the present invention is defined by the appended claims and equivalents thereto.

What is claimed is:

1. A colorimetric based gastric tube placement verification system for a patient gastric tube comprising:
a) A housing configured to be coupled to the gastric whereby stomach content aspirate can flow through an internal passage of the housing;
b) At least one colorimetric based sensor within the housing and configured to come into contact with the patient stomach content aspirate, the least one colorimetric based sensor configures to detect a first gastric acid.

2. The colorimetric based gastric tube placement verification system according to claim 1, wherein at least two colorimetric based sensors are present forming a colorimetric chemical sensor array.

3. The colorimetric based gastric tube placement verification system according to claim 2, wherein one sensor senses butyric acid.

4. The colorimetric based gastric tube placement verification system according to claim 3, wherein one colorimetric sensor senses hydrochloric acid.

5. The colorimetric based gastric tube placement verification system according to claim 4, wherein the housing is transparent.

6. The colorimetric based gastric tube placement verification system according to claim 5, wherein the housing includes an aspiration syringe barrel.

7. The colorimetric based gastric tube placement verification system according to claim 1, wherein at least one sensor senses butyric acid.

8. The colorimetric based gastric tube placement verification system according to claim 7, wherein the housing is transparent.

9. The colorimetric based gastric tube placement verification system according to claim 8, wherein the housing includes an aspiration syringe barrel.

10. The colorimetric based gastric tube placement verification system according to claim 1, wherein at least one sensor senses hydrochloric acid.

11. The colorimetric based gastric tube placement verification system according to claim 10, wherein the housing is transparent.

12. The colorimetric based gastric tube placement verification system according to claim 11, wherein the housing includes an aspiration syringe barrel.

13. A colorimetric based gastric tube placement verification system for a patient gastric tube comprising:
a) A housing configured to be coupled to the gastric whereby stomach content aspirate can flow through an internal passage of the housing;
b) A colormetric sensor array comprising at least two colorimetric based sensors within the housing and configured to come into contact with the patient stomach content aspirate, including i) a sensor for a first gastric acid, and ii) a sensor for a second gastric acid different from the first gastric acid.

14. The colorimetric based gastric tube placement verification system according to claim 13, wherein one sensor senses butyric acid.

15. The colorimetric based gastric tube placement verification system according to claim 14, wherein one sensor senses hydrochloric acid.

16. The colorimetric based gastric tube placement verification system according to claim 15, wherein the housing is transparent.

17. The colorimetric based gastric tube placement verification system according to claim 13, wherein one sensor senses hydrochloric acid.

18. A method of gastric tube placement verification comprising the steps of:
a) Inserting a gastric tube within the patient;
b) Providing a colorimetric based gastric tube placement verification system for a patient gastric tube including i) a housing configured to be coupled to the gastric tube whereby stomach content aspirate can flow through an internal passage of the housing; and ii) at least one colorimetric based sensor within the housing and configured to come into contact with the patient stomach content aspirate, the least one colorimetric based sensor configures to detect a first gastric acid;
c) Coupling the housing of the colorimetric based gastric tube placement verification system to a proximal end of the gastric tube;
d) Aspirating stomach content of the patient whereby stomach aspirate can flow through an internal passage of the housing; and
e) Visually inspecting at least one colorimetric based sensor within the housing for verification of proper gastric tube placement.

19. The method of gastric tube placement verification according to claim 18, wherein at least two colorimetric based sensors are present forming a colorimetric chemical sensor array.

20. The gastric tube placement verification according to claim 19, wherein one sensor senses butyric acid, and wherein one colorimetric sensor senses hydrochloric acid.
